(19) United States
(12) Patent Application Publication
(10) Pub. No.: US 2016/0295126 A1
(43) Pub. Date: Oct. 6, 2016

(54) Image Stitching with Local Deformation for in vivo Capsule Images

(71) Applicant: Capso Vision, Inc., Saratoga, CA (US)

(72) Inventors: Kang-Huai Wang, Saratoga, CA (US); Chenyu Wu, Sunnyvale, CA (US)

(73) Assignee: Capso Vision, Inc.

(21) Appl. No.: 14/678,894

(22) Filed: Apr. 3, 2015

(51) Int. Cl. H04N 5/265; A61B 1/04; H04N 7/18

(52) U.S. Cl. CPC H04N 5/265; H04N 7/183; A61B 1/041; H04N 2005/2255

(57) ABSTRACT

A method of processing images captured using an in vivo capsule camera is disclosed. Input images captured by the in vivo capsule camera are received and used as to-be-processed images. At least one locally-deformed stitched image is generated by applying local deformation to image areas in a vicinity of a seam between two to-be-processed images and stitching the two locally deformed to-be-processed images. Output images including the at least one locally-deformed stitched image are provided for display or further processing. The process to generate at least one locally-deformed stitched image may comprise identifying an optimal seam between the two to-be-processed images and applying the local deformation to the image areas in the vicinity of the optimal seam. The process of identifying the optimal seam comprises minimizing differences of an object function across the optimal seam. The object function may correspond to image intensity or derivative of the image intensity.

### CROSS REFERENCE TO RELATED APPLICATIONS

The present invention is related to PCT Patent Application, Ser. No. PCT/US14/38533, entitled “Reconstruction of Images from an in vivo Multi-Cameras Capsule”, filed on May 19, 2014. The PCT Patent Application is hereby incorporated by reference in its entirety.

### FIELD OF THE INVENTION

The present invention relates to image stitching from images captured using in vivo capsule camera and their display thereof. In particular, the present invention uses local deformation in the vicinity of stitched images to avoid large image distortion after a large number of images are stitched.

### BACKGROUND AND RELATED ART

Capsule endoscope is an in vivo imaging device which addresses many of problems of traditional endoscopes. A camera is housed in a swallowable capsule along with a radio transmitter for transmitting data to a base-station receiver or transceiver. A data recorder outside the body may also be used to receive and record the transmitted data. The data primarily comprises images recorded by the digital camera. The capsule may also include a radio receiver for receiving instructions or other data from a base-station transmitter. Instead of using radio-frequency transmission, lower-frequency electromagnetic signals may be used. Power may be supplied inductively from an external inductor to an internal inductor within the capsule or from a battery within the capsule. In another type of capsule camera with on-board storage, the captured images are stored on-board instead of transmitted to an external device. The capsule with on-board storage is retrieved after the excretion of the capsule. The capsule with on-board storage provides the patient the comfort and freedom without wearing the data recorder or being restricted to proximity of a wireless data receiver.

While forward-looking capsule cameras include one camera, there are other types of capsule cameras that use multiple cameras to provide side view or panoramic view. A side or reverse angle is required in order to view the tissue surface properly. It is important for a physician or diagnostician to see all areas of these organs, as polyps or other irregularities need to be thoroughly observed for an accurate diagnosis. A camera configured to capture a panoramic image of an environment surrounding the camera is disclosed in U.S. patent application Ser. 11/642,275, entitled “In vivo sensor with panoramic camera” and filed on Dec. 19, 2006.

In an autonomous capsule system, multiple images along with other data are collected during the course when the capsule camera travels through the gastrointestinal (GI) tract. The images and data after being acquired and processed are usually displayed on a display device for a diagnostician or medical professional to examine. However, each image only provides a limited view of a small section of the GI tract. It is desirable to form a large picture from multiple capsule images representing a single composite view. For example, multiple capsule images may be used to form a cut-open view of the inner GI tract surface. The large picture can take advantage of the high-resolution large-screen display device to allow a user to visualize more information at the same time. The image stitching process may involve removing the redundant overlapped areas between images so that a larger area of the inner GI tract surface can be viewed at the same time as a single composite picture. In addition, the large picture can provide a complete view or a significant portion of the inner GI tract surface. It should be easier and faster for a diagnostician or a medical professional to quickly spot an area of interest, such as a polyp.

In the field of computational photography, image mosaicking techniques have been developed to stitch smaller images into a large picture. A review of general technical approaches to image alignment and stitching can be found in “Image Alignment and Stitching: A Tutorial”, by Szeliski, Microsoft Research Technical Report MSR-TR-2004-92, Dec. 10, 2006.

The feature-based matching first determines a set of feature points in each image and then compares the corresponding feature descriptors. To match two image patches or features captured from two different viewing angles, a rigid model including scaling, rotation, etc. is estimated based on the correspondences. To match two images captured deforming objects, a non-rigid model including local deformation can be computed.

The number of feature points is usually much smaller than the number of pixels of a corresponding image. Therefore, the computational load for feature-based image matching is substantially less that for pixel-based image matching. However, it is still time consuming for pair-wise matching. Usually k-d tree, a well-known technique in this field, is utilized to speed up this procedure. Accordingly, feature-based image matching is widely used in the field. Nevertheless, the feature-based matching may not work well for images under some circumstances. In this case, the direct image matching can always be used as a fall back mode, or a combination of the above two approaches may be preferred.

Image matching techniques usually assume certain motion models. When the scenes captured by the camera consist of rigid objects, image matching based on either feature matching or pixel domain matching will work reasonably well. However, if the objects in the scene deform or lack of distinguishable features, it makes the image matching task very difficult. For capsule images captured during the course of travelling through the GI track, the situation is even more challenging. Not only the scenes corresponding to walls of the GI track deform while camera is moving, but also the scenes are captured with a close distance from the camera and often are lack of distinguishable features. Due to the close distance between objects and the camera, the often used camera models may fail to produce good match between different scenes. Also, light reflection from near objects may cause over exposure for some parts of the object. In addition, when a large number of images are stitched, the distortion may accumulate and causes distortion grow larger and larger. Therefore, it is desirable to develop methods that can overcome these issues mentioned.

### SUMMARY OF INVENTION

A method of processing images captured using an in vivo capsule camera is disclosed. A plurality of input images captured by the in vivo capsule camera are received and used as to-be-processed images. At least one locally-deformed stitched image is generated by applying local deformation to image areas in a vicinity of a seam between two to-be-processed images and stitching the two locally deformed to-be-processed images. One or more output images including said at least one locally-deformed stitched image are provided for display or further processing.

The process to generate at least one locally-deformed stitched image comprises identifying an optimal seam between the two to-be-processed images and applying the local deformation to the image areas in the vicinity of the optimal seam. The process of identifying the optimal seam may comprise minimizing differences of an object function across the optimal seam. The object function may correspond to image intensity or derivative of the image intensity.

The to-be-processed images may correspond to pairwise-stitched images derived from the plurality of input images, where each pairwise-stitched image is formed by deforming two neighboring images of the plurality of input images and stitching said two neighboring images. The to-be-processed images may correspond to individual images of the plurality of images. The to-be-processed images may also correspond to short-stitched images of the plurality of images, where each short-stitched image is formed by deforming a small number of images and stitching the small number of images.

The process of generating said at least one locally-deformed stitched image may comprise two separate processing steps, where the first step corresponds to applying the local deformation to the image areas in the vicinity of the seam between the two to-be-processed images and the second step corresponds to said stitching the two to-be-processed images locally deformed. The first step and the second step can be performed iteratively. The first step and the second step can be terminated after a pre-defined number of iterations. Alternatively, the first step and the second step can be terminated when a stop criterion is met. The stop criterion may be triggered if the seam in a current iteration is the same as or substantial the same as the seam in a previous iteration.

Multiple locally-deformed stitched images can be generated from the to-be-processed images by sequentially stitching a next input image to a current stitched image starting from a beginning input image corresponding to a smallest time index. Multiple locally-deformed stitched images can be generated from the to-be-processed images by sequentially stitching a next input image with a current stitched image starting from a last input image corresponding to a largest time index. Multiple locally-deformed stitched images can also be generated from the to-be-processed images by sequentially stitching one next input image with one current stitched image starting from an intermediate input image to a last image, and sequentially stitching one next input image to one current stitched image starting from the intermediate input image to a beginning image, where the intermediate input image has an intermediate time index between a smallest time index and a largest time index.

The process of generating at least one locally-deformed stitched image may comprise applying the local deformation to the image areas in the vicinity of a next seam between a next image and a currently stitched image and stitching the next image and the currently stitched image. The image area associated with the currently stitched image in the vicinity of the next seam may correspond to a minimum area bounded by the next seam, one or more previous seams of the currently stitched image, and natural image boundary of the currently stitched image.

### BRIEF DESCRIPTION OF THE DRAWINGS

FIG. 1 illustrates an exemplary image stitching with local deformation according to an embodiment of the present invention, where a minimum area bounded by a current optimal seam, one or more previous optimal seams and the boundary of the image being stitched.

FIG. 2 illustrates an exemplary flowchart of a system for image stitching with local deformation according to an embodiment of the present invention.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely a representative of selected embodiments of the invention. References throughout this specification to “one embodiment,” “an embodiment”, or similar language mean that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention. The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

As mentioned before, image matching may not work well for images under some circumstances, particularly for images captured using a capsule image travelling through the human gastrointestinal (GI) track. For images corresponding to natural scenes captured using a digital camera, image mosaicking or stitching usually works reasonable well. The process usually involves image registration among multiple images. After registration is done and image model parameters are derived, images are warped or deformed based on a reference picture. The images are then blended to form one or more stitched image. For natural scenes, image models usually work reasonably well since there are distinct features in the scenes and also there are large stationary backgrounds. Nevertheless, the images from the gastrointestinal (GI) tract present a very challenging environment for image stitching due to various reasons such as the lack of features in the scenes, contraction and relaxation of the GI tract, etc. Furthermore, the images captured from the GI tract during the course of imaging are in the order over tens of thousands. When the large number of images is warped to a reference image, the distortion may accumulate and the registration quality for the image far away from the reference image may become very poor. Therefore, it is desirable to develop a technique that can stitch images, such as images of GI tract with non-ideal models.

Since the GI tract may deform locally along time, stitching multiple images across a long time period together requires substantially deforming images that are far away (time domain) from the reference image frame. This may cause large distortion to those images and make parts of the final stitched image unreadable. In order to deal with this issue, embodiments of the present invention disclose an alternative representation of the final stitched image including locally stitched images corresponding to different time stamps. For example, there are n images, i₁, i₂, i₃, . . . , iₙ to be stitched. Every two adjacent images can be stitched together first. Therefore, images i₁ and i₂ can be stitched to form i(1,2). Images i₂ and i₃ can be stitched to form i(2,3), etc. At the end, stitched images i(1,2), i(2,3), i(3,4), . . . , i(n−1,n) are formed. In the new list of images, each pair of adjacent images includes a common image in a non-deformed or deformed format. For example, the pair of images i(1,2) and i(2,3) include i₂ or deformed i₂. Assuming that the first image is always used as an initial local reference image, image i₂ is deformed in i(1,2) and the deformed i₂ corresponds to what it should look like at time t₁. Image i₂ in i(2,3) is not deformed. Similarly, image i₃ is deformed in i(2,3) and the deformed i₃ corresponds to what it should look like at time i₂ (i.e., i₂ being a local reference picture). To avoid accumulated deformation across images, stitching a large number of images should be avoided. For example, after forming stitched images i(1,2) and i(2,3), the two stitched images will not be furthered stitched using regular stitching. Instead, an optimal seam between deformed i₂ and non-deformed i₂ is determined and the two images are blended accordingly. Accordingly, multiple pairwise stitched images representing different time stamps can be blended into a big picture. When this stitched picture is viewed from the left to the right, it will be similar to look at a video from time i₁ to time iₙ, without substantial distortion.

While blending two images without registration, the two pairwise stitched images may be misaligned. On the other hand, if image registration is applied to the two pairwise stitched images, the distortion will accumulate. When a large number of pairwise stitched images are stitched, the accumulated distortion will become substantial. Furthermore, the computational loading for stitching a large image is substantial. In order to overcome these issues, an embodiment of the present invention identifies an optimal seam between two images and deforms only the image area in the vicinity of the optimal seam. For example, stitching images i(1,2) and i(2,3) according to an embodiment of the present invention will deform i(1,2) or i(2,3) or both locally in the vicinity of the optimal seam to generate a natural look around the seam. Before finding the optimal seam, a rigid transformation may be applied to two to-be-stitched images. In the optimal seam process, an object function is used for deriving the optimal seam. For a selected object function, the optimal seam is determined such that the differences along the optimal seam are minimized. For example, the object function may correspond to the intensity function of the image or the derivative of the intensity function. Accordingly, the optimal seam may be derived to minimize the differences of the intensities at both side of the boundary or the differences of derivative of the intensities at both side of the boundary. With the differences minimized across the optimal seam, the stitched image will look smooth along the seam.

The stitching with local deformation process as disclosed above can choose the initial reference time as t₁, i.e., the first time index. The initial reference time index can be also set to the last index, t_{N}. Therefore, i(N, N−1) is based on t_{N}, i(N−1, N−2) is based on t_{(N-1)}, etc. The initial reference time index may also be set to t_{M}, where t₁<t_{M}<t_{N}, and the process starts from this inside time point toward both ends. In other words, the process will start from t_{M} toward t₁ to deform i(M, M−1) based t_{M}, i(M−1, M−2) based on t_{(M-1)}, and from t_{M} toward t_{N} to deform t(M, M+1) based on t_{M}, i(M+1, M+2) based on t_{(M+1)}, etc. In the above description, image i_{M} can be stitched both to the right image and to the left image, so there are both i(M−1,M) and i(M, M+1).

In the above example, the stitching with local deformation process is applied to pairwise-stitched images. Nevertheless, the process can also be applied to individual images, i.e., i₁, i₂, . . . , i_{N}. For example, after i₁ and i₂ are stitched with local deformation to form i(1,2), i(1,2) is to be stitched to the next image, i₃. In this manner, the currently stitched i(1, 2, 3, . . . , N−1) is to be stitched to the next image, i_{N}. In this case, only the newly incrementally stitched image, i_{N} or both sides of the optimal seam will be deformed. If both sides are deformed, on the i(1, 2, 3, . . . , N−1) side, only the portion between the new seam and the seam between i(1, 2, 3, . . . , N−2) and i_{(N-1}), i.e. a previous seam corresponding to the last stitching operation will be deformed. This will avoid the need for deforming a very large portion of i(1, . . . , N−1). For example, there are 100,000 images in one capsule procedure and a currently stitched image i(1, . . . ,90000) is to be stitched with a next image i(90001), which would require to deform the entire currently stitched image i(1, . . . ,90000) and this would be impractical. In this case, the locus of the seam of the last stitching operation and an object function are set as boundary conditions, where the object function corresponds to the intensity function or the derivative of the intensity function. Therefore, the deformation can be applied all the way to the last M seams, while the newest (M−1) seams keep maintained fixed or as aforementioned could be optimized as to intensity function and/or first derivative of the intensity function.

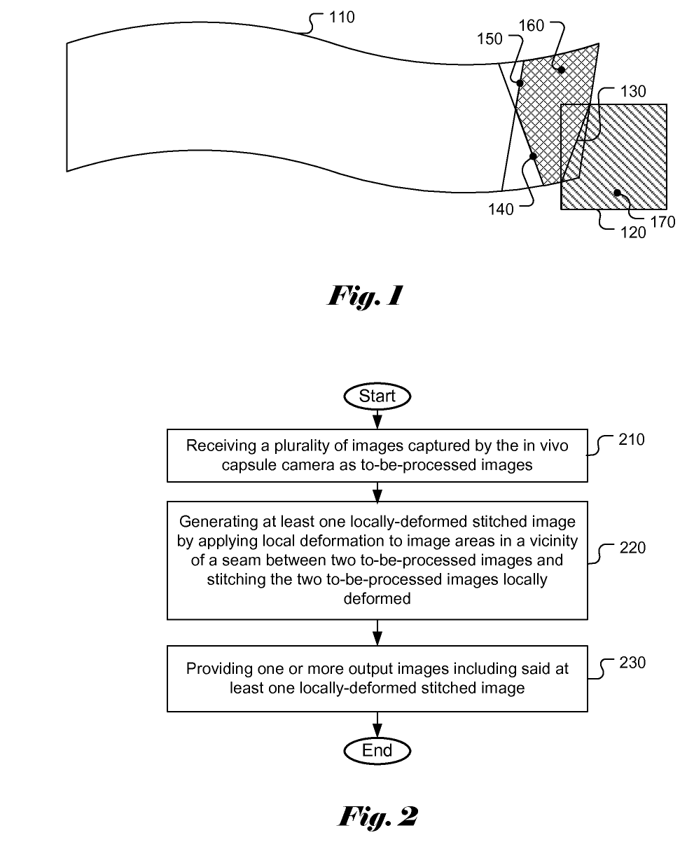

As mentioned above, to deform a large image would be a heavy computational burden. The present invention overcomes the issue by limiting the deformation to the vicinity of the optimal seam. According to an embodiment of the present invention, an area of the previously stitched image adjacent to the optimal seam is identified and the local deformation is applied to this area. FIG. 1 illustrates an example of the areas subject to local deformation. The currently stitched image 110 and a next image 120 are to be stitched. An optimal seam 130 between image 110 and image 120 is determined. Image 110 contains a previous seam 140 and a further previous seam 150. The minimum area bounded by the seams (130, 140 and 150) and the boundary of image 110 is identified and shown by the area 160. For image 120, the area subject to local deformation is identified and shown by area 170. For image 110, area 160 to be deformed is much smaller than the area of the entire image. Consequently, the required computations are substantially reduced. On the other hand, the stitched image between image 110 and image 120 has smooth transition from one image to another.

In the above example, the stitching with local deformation process is applied to a next image to stitch with a large image generated by the same stitching with local deformation process. Also, examples of stitching with local deformation have been illustrated for stitching pairwise-stitched images and individual images. The present invention may also be applied to images, where each image corresponds to a small number of images stitched using conventional stitching techniques. As long as the number of stitched images is not large, the distortion may be limited. Therefore, the present invention may be applied to these pre-stitched images to form a large image without the issue of accumulated distortion.

During the process of identifying the optimal seam and performing local deformation, an object function is selected and image model for deformation are derived so as to minimize the differences of the object function along the seam. In other words, the optimal seam is determined at the same time as the image model for deformation is derived. In another embodiment, the process for seam determination and the process of local deformation can be separate. For example, an initial seam can be determined without any local deformation. After the initial seam is determined, local deformation is applied in the vicinity of the seam. The seam can be refined after local deformation. The process of seam determination and the process of local deformation can be applied iteratively. The process can be terminated after a pre-defined number of iterations. Alternatively, the process can be terminated when a stop criterion is triggered. For example, when the seam in the current iteration is the same as or substantial the same as that in the previous iteration, the process can be terminated.

FIG. 2 illustrates an exemplary flowchart of a system for image stitching with local deformation according to an embodiment of the present invention. A plurality of images captured by the camera is received as shown in step 210. The images may be retrieved from memory or received from a processor. At least one locally-deformed stitched image is generated by applying local deformation to image areas in a vicinity of a seam between two to-be-processed images and stitching the two to-be-processed images locally deformed in step 220. One or more output images including said at least one locally-deformed stitched image are provided for display or further processing in step 230.

The invention may be embodied in other specific forms without departing from its spirit or essential characteristics. The described examples are to be considered in all respects only as illustrative and not restrictive. Therefore, the scope of the invention is indicated by the appended claims rather than by the foregoing description. All changes which come within the meaning and range of equivalency of the claims are to be embraced within their scope.

1. A method of processing images captured using an in vivo capsule camera, the method comprising:
receiving a plurality of input images captured by the in vivo capsule camera as to-be-processed images;
generating at least one locally-deformed stitched image by applying local deformation to image areas in a vicinity of a seam between two to-be-processed images and stitching the two to-be-processed images locally deformed; and
providing one or more output images including said at least one locally-deformed stitched image.

2. The method claim 1, wherein said generating said at least one locally-deformed stitched image comprises identifying an optimal seam between the two to-be-processed images and applying the local deformation to the image areas in the vicinity of the optimal seam.

3. The method claim 2, wherein said identifying the optimal seam comprises minimizing differences of an object function across the optimal seam.

4. The method claim 3, wherein the object function corresponds to image intensity or derivative of the image intensity.

5. The method claim 1, wherein the to-be-processed images comprise pairwise-stitched images derived from the plurality of input images, wherein each pairwise-stitched image is formed by deforming two neighboring images of the plurality of input images and stitching said two neighboring images.

6. The method claim 1, wherein the to-be-processed images comprise individual images of the plurality of images.

7. The method claim 1, wherein the to-be-processed images comprise short-stitched images of the plurality of images, wherein each short-stitched image is formed by deforming a small number of images and stitching the small number of images.

8. The method claim 1, wherein said generating said at least one locally-deformed stitched image comprises two separate processing steps, wherein a first step corresponds to said applying the local deformation to the image areas in the vicinity of the seam between the two to-be-processed images and a second step corresponds to said stitching the two to-be-processed images locally deformed.

9. The method claim 8, wherein the first step and the second step are performed iteratively.

10. The method claim 9, wherein the first step and the second step are terminated after a pre-defined number of iterations.

11. The method claim 9, wherein the first step and the second step are terminated when a stop criterion is met.

12. The method claim 11, wherein the stop criterion is triggered if the seam in a current iteration is the same as or substantial the same as the seam in a previous iteration.

13. The method claim 1, wherein at least one locally-deformed stitched images are generated from the to-be-processed images by sequentially stitching a next input image to a current stitched image starting from a beginning input image corresponding to a smallest time index.

14. The method claim 1, wherein at least one locally-deformed stitched images are generated from the to-be-processed images by sequentially stitching a next input image with a current stitched image starting from a last input image corresponding to a largest time index.

15. The method claim 1, wherein at least one locally-deformed stitched images are generated from the to-be-processed images by sequentially stitching one next input image with one current stitched image starting from an intermediate input image, and sequentially stitching one next input image to one current stitched image starting from the intermediate input image, wherein the intermediate input image has an intermediate time index between a smallest time index and a largest time index.

16. The method claim 1, wherein said generating at least one locally-deformed stitched image comprises applying the local deformation to the image areas in the vicinity of a next seam between a next image and a currently stitched image and stitching the next image and the currently stitched image.

17. The method claim 16, wherein the image area associated with the currently stitched image in the vicinity of the next seam corresponds to a minimum area bounded by the next seam, one or more previous seams of the currently stitched image, and natural image boundary of the currently stitched image.

18. A system for processing images captured using an in vivo capsule camera, the system comprising:
a first processing unit configured to receive a plurality of input images captured by the in vivo capsule camera as to-be-processed images;
a second processing unit configured to generate at least one locally-deformed stitched image by applying local deformation to image areas in a vicinity of a seam between two to-be-processed images and stitching the two to-be-processed images locally deformed; and
a third processing unit configured to provide one or more output images including said at least one locally-deformed stitched image.
